(19) United States
(12) Patent Application Publication
(10) Pub. No.: US 2008/0091190 A1
(43) Pub. Date: Apr. 17, 2008

(54) Injectable surgical patch and method for performing same

(75) Inventors: John D. Carlton, Las Vegas, NV (US); J. Bruce Dunne, Longmont, CO (US)

Correspondence Address: COVIDIEN, 60 MIDDLETOWN AVENUE, NORTH HAVEN, CT, 06473, US

(73) Assignee: TYCO Healthcare Group LP

(21) Appl. No.: 11/581,867

(22) Filed: Oct. 17, 2006

(51) Int. Cl. A61B 18/18

(52) U.S. Cl. 606/41

(57) ABSTRACT

A system for excision of abnormal tissue includes a sealant material having an uncured configuration which allows the sealant material to be readily injected between target tissue and underlying tissue and a cured configuration wherein the sealant material acts as a substantially solid barrier between the target tissue and underlying tissue. The sealant material acts to initially separate or lift the target tissue relative to the underlying tissue to facilitate excision of the target tissue via a snare, wire loop, forceps, knife, scissors and/or combinations thereof. After excision of the target tissue, the sealant acts to seal the underlying tissue from the surrounding environment.

### BACKGROUND

The present disclosure relates generally to devices and methods for use with surgical resection and, more particularly, the present disclosure relates to an injectable surgical patch for use with endoscopic surgical resection such as endoscopic mucosal resection (EMR).

### TECHNICAL FIELD

As an alternative to open forceps for use with open surgical procedures, many modern surgeons use endoscopes and endoscopic instruments for remotely accessing organs or cavities through natural orifices such as the mouth or anus without the need for incisions. As a direct result thereof, patients tend to benefit from less scarring and reduced healing time.

Endoscopic surgical instruments are inserted into the patient through a natural body orifice to the treatment site. Improved endoscopic techniques have enabled the diagnosis and removal of many large polyps or early cancers, which previously required an open surgical approach.

Recent developments have changed the operational mechanism and design of endoscopes and the accessory apparatus and clinical applications using this equipment are evolving. For example, in gastrointestinal applications, endoscopic treatments such as endoscopic mucosal resection, percutaneous endoscopic gastrostomy (PEG), and stent placement are increasingly applied. Endoscopic mucosal resection (EMR), which may be utilized to conserve the esophagus, stomach or bowel, is a minimally invasive and attractive therapeutic modality for early stage cancers. EMR is a relatively new endoluminal therapeutic technique that is particularly advocated for the treatment of Barrett's esophagus (BE)-related superficial neoplasms, stomach abnormalities and bowel lesions or polyps. EMR is based on the concept that endoscopy provides visualization and access to the mucosa, the innermost lining of the tissue tract, e.g., the gastrointestinal tissue tract where most gastrointestinal cancers from the esophagus to the rectum have their origin. The EMR surgical technique combines the therapeutic power of endoscopic surgery with the diagnostic power of pathology examination of resected tissue.

Most gastrointestinal or colon-type cancers arise in mucosal polyps, which project into the lumen of the colon, making them relatively easy to remove by endoscopy using wire loops or snares to grasp the polyp base. The polyps are then excised with electric current, producing simultaneous cutting action and cauterization. In the stomach, however, most cancers do not begin in polyps, but rather in only slightly elevated, flat, or slightly depressed mucosal dysplastic lesions. Such lesions are very difficult to grasp with a simple wire loop or snare. Various endoscopists have adapted and perfected a number of methods to elevate the diseased mucosal area so that snaring would be possible. Most of these techniques use fluid injection into the submucosa, the layer of the gastrointestinal tract immediately below the mucosa, to elevate the mucosa and allow it to be grasped with a wire loop or snare. Fluid may also be injected between the two layers to accomplish the same purpose. The success of EMR in the stomach prompted endoscopists to expand the use of the method to the esophagus, where early cancer and pre-malignant dysplasia also tends to be nonpolypoid and flat, and also to the colon, where it can be used to assist in removal of both small and large flat or sessile polyps.

### SUMMARY

The present disclosure relates to a system for excision of abnormal tissue which includes a sealant material having an uncured configuration allowing the sealant material to be readily injected between target tissue and underlying tissue and a cured configuration wherein the sealant material acts as a substantially solid barrier between the target tissue and underlying tissue. The sealant material initially acts to separate the target tissue relative to the underlying tissue to facilitate excision of the target tissue via a snare, wire loop, forceps, knife and/or scissors and combinations thereof. After excision of the target tissue, the sealant material acts to seal the underlying tissue from the surrounding environment, such as stomach, esophageal or bowel contents, bacteria or other harmful or infectious elements. The sealant material may also act to provide hemostasis since some excised polyps may continue to bleed for prolonged periods of time. For example, a coagulant may be included in the formulation of the sealant material to accomplish this purpose.

In one envisioned embodiment, during transition from the uncured configuration to the cured configuration, the sealant material expands to separate the target tissue from the underlying tissue. The sealant material may be curable upon selective application of temperature, electrosurgical energy, ultrasonic energy, light and/or combinations thereof. The sealant material may also be particularly formulated to provide thermal insulation for adjacent tissue layers during electrical activation or curing depending upon a particular purpose.

In yet another embodiment according to the present disclosure, the sealant material may be formulated to include collagen, liposomes, elastin and/or combinations thereof. The sealant material may also be formulated to include an antibiotic solution, an antibacterial solution, antibodies, hemostatic solution and/or combinations thereof. In one particularly advantageous embodiment, the sealant material is formulated to include collagen which is designed to react or cooperate with native or existing collagen to form a high strength seal.

The sealant may also be formulated to include a material which regulates the sealant's impedance during excision (i.e., the application of energy) which, in turn, regulates the temperature of surrounding tissue layers.

The present disclosure also relates to a system for excision of abnormal tissue during endoscopic mucosal resection (EMR) which includes a sealant material having an uncured configuration and a cured configuration. The uncured configuration allows the sealant material to be readily injected through an endoscopic instrument adjacent or proximate abnormal tissue and between the mucosa tissue layer and submucosa tissue layer. When cured, the sealant material acts as a substantially solid barrier between the mucosa tissue layer and submucosa tissue layer. The sealant material, when injected, separates the abnormal tissue and mucosa tissue layer relative to the submucosa tissue layer to facilitate excision of the abnormal tissue via at least one of a snare, wire loop, forceps, knife, scissors and/or combinations thereof. After excision of the abnormal tissue, the sealant material essentially protects or seals the submucosa tissue layer from the surrounding environment. During the transition from the uncured to cured configuration, the sealant material may expand to separate (or further separate) the mucosa tissue layer from the submucosa tissue layer.

The present disclosure also relates to a method for excising abnormal tissue and includes the steps of: providing a sealant material having an uncured configuration allowing the sealant material to be readily injected between target tissue and underlying tissue and a cured configuration wherein the sealant material acts as a substantially solid barrier between the target tissue and underlying tissue; injecting the sealant material between the target tissue and the underlying tissue to initially separate the target tissue relative to the underlying tissue; allowing the sealant material to cure; and excising the target tissue utilizing a snare, wire loop, forceps, knife, scissors and/or combinations thereof. After excision of the target tissue, the sealant material acts to seal the underlying tissue from the surrounding environment.

The sealant material of the providing step may be formulated to expand during the transition from an uncured configuration to a cured configuration. The step of curing the sealant material may include the step of selectively applying temperature, electrosurgical energy, ultrasonic energy, light and/or combinations thereof to cure the sealant material.

The sealant material of the providing step may be formulated to include collagen, liposomes, elastin and/or combinations thereof. The sealant material may also be formulated to include an antibiotic solution, an antibacterial solution, antibodies, hemostatic solution and/or combinations thereof. Moreover, the sealant material may be formulated to include a material which regulates the sealant's impedance during excision (i.e., the application of energy) to regulate the temperature of surrounding tissue layers.

The present disclosure also relates to a method for excising abnormal tissue and includes the steps of: providing a substantially liquid sealant material operably transitionable between an uncured configuration and a cured configuration when exposed to temperature, electrosurgical energy, ultrasonic energy and/or light. The substantially liquid sealant material is formulated with an antibiotic solution, an antibacterial solution, hemostatic solution and/or antibodies. The method also includes the steps of injecting the substantially liquid sealant material in an uncured configuration between target tissue and underlying tissue to initially separate the target tissue relative to the underlying tissue and allowing the substantially liquid sealant material to transition from the uncured configuration to the cured configuration by selectively exposing the substantially liquid sealant material to temperature, electrosurgical energy, ultrasonic energy and/or light. The target tissue is then excised whereafter the sealant material acts to seal the underlying tissue.

### BRIEF DESCRIPTION OF THE DRAWINGS

Various embodiments of the subject instrument are described herein with reference to the drawings wherein:

FIG. 1 is schematic representation of an abnormal tissue growth or polyp disposed in a mucosa layer of a gastrointestinal tissue tract;

FIG. 2 is schematic representation of a patch material according to the present disclosure being injected or disposed into a submucosal tissue layer beneath the abnormal tissue growth to separate the tissue layers for excision or resection purposes;

FIG. 3 is schematic representation of a wire loop or snare encircling the abnormal tissue growth for excision or resection purposes;

FIG. 4 is schematic representation showing the abnormal tissue growth separated from the mucosal tissue layer after excision or resection; and

FIG. 5 is a schematic representation of the abnormal tissue growth being removed from the treated tissue site while the patch material seals the site from the surrounding environment.

### DETAILED DESCRIPTION

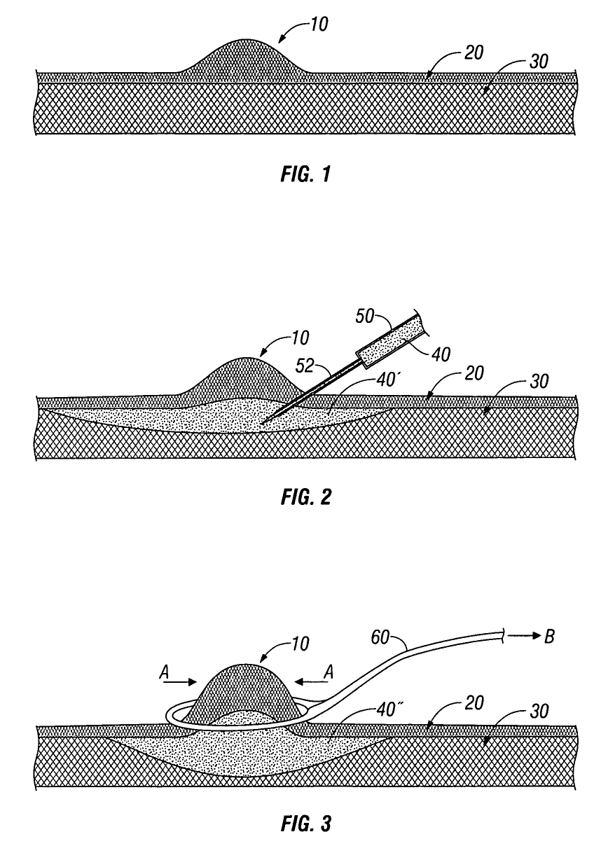

Turning now in detail to FIGS. 1-5, one embodiment of the present disclosure includes a patch 40 that may be utilized to facilitate resection of abnormal tissue structures, such as polyps and other tissue defects. More particularly, and as best shown in FIG. 1, a typical abnormal tissue defect 10 normally occurs in the mucosa layer 20 of a tissue membrane. Below the mucosa lies the submucosa 30.

Endoscopic mucosal resection (EMR) is a promising technique for local management of mucosal cancer, e.g., Barrett's high grade dysplasia (HGD). Recent studies have indicated that different techniques of EMR are feasible and safe for this indication. The procedure can be used to obtain large biopsies for diagnosis and local tumor staging. Local complete remission can be achieved in the majority of patients with focal lesions so that EMR is potentially curative provided that there is no evidence of submucosal tumor infiltration.

Several endoscopic techniques have been advocated in the past for mucosal resection. It is difficult to compare the efficacy and safety among the various techniques. For example, one of the easiest but not necessarily the most effective method involves simply snaring tissue by use of a stiff snare and applying suction to remove the abnormal tissue once resected. A so-called “polypectomy technique” involves initially injecting a solution, e.g., an epinephrine solution, into the submucosal layer to facilitate snaring and resection of the abnormal tissue lesions. Other techniques such as a “lift and cut technique” involve dual-channel endoscopes: a biopsy forceps is inserted through the second endoscopic channel to lift the lesion into the opened snare which is subsequently closed around the lesion. Still other known techniques involve the creation of an artificial polyp at the site by suction, which allows a polypectomy snare to be positioned around the lesion for resection purposes. A variety of other methods involve the use of so-called overtubes that provide an external instrumentation channel for insertion of forceps and snares. This technique allows resection of a large specimen but has been associated with an increased risk of perforation.

Another popular technique called “Endoscopic mucosal resection cap (EMRC) procedure” is performed with a specially designed thin snare that is pre-looped in a cap mounted on the tip of a forward-view endoscope. To decrease the potential risk of perforation, saline solution is injected into the submucosal layer to separate the mucosa from the muscle layer. The mucosal lesion is sucked into the cap and firmly strangulated by the snare wire. The polyp is resected by electrocautery and removed by suction. It is also common to separate the submucosa and mucosa layers from muscle by injecting a solution between the submucosa and the muscle. Moreover, some procedures also involve excising both mucosa and submucosa layers, e.g., polypectomy procedure.

The presently disclosed patch 40 and surgical technique may be suited for endoscopic mucosa resection type procedures which, as described above, involves the resection of a portion of the mucosal tissue layer to remove an abnormal tissue lesion or polyp via the use of an endoscope. As best shown in FIG. 2, a needle 50 is configured to retain a sealant material 40′ for injection into the submucosal tissue layer 30 via needle tip 52. In one embodiment, the sealant material 40′ is adapted to transition from an uncured configuration, wherein the sealant material 40′ is allowed to be injected into the submucosal tissue layer 30 proximate the target or abnormal tissue growth 10 in the mucosal tissue layer 20, to a cured configuration, wherein the sealant material 40′ acts as a substantially solid barrier between the abnormal tissue growth 10 and the underlying tissue 30. During infusion or injection, and as the sealant material 40′ transitions from an uncured injectable configuration 40′ to a cured configuration 40″ (FIG. 3), the sealant material 40′ separates and/or lifts the abnormal tissue growth 10 relative to the underlying submucosal tissue 30 to facilitate excision of the abnormal tissue growth 10 via a snare 60 or other suitable device.

As best shown in FIG. 3, the snare 60 is utilized to encircle and strangulate the abnormal tissue growth 10 in the direction of arrow “A” by manipulating the snare 60 in the direction of arrow “B”. The snare may be energized and/or include sharpened elements to excise the growth 10. Other suitable resection or excision instruments (not shown) may also be used to remove the abnormal tissue growth, e.g., wire loop, forceps, knife, scissors and combinations thereof.

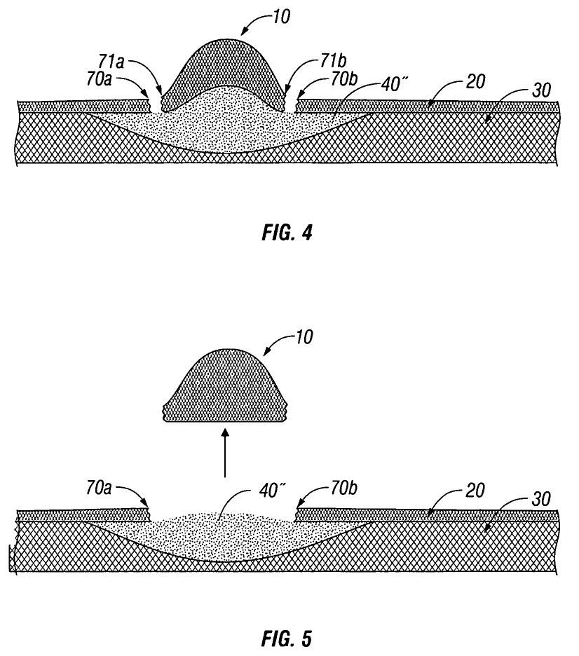

As best shown in FIG. 4, after excision of the abnormal tissue growth 10, the growth 10 is removed from the treated tissue site by suction, irrigation or other suitable surgical cleansing techniques. Once the abnormal tissue growth 10 is removed, the cured sealant material 40″ acts to seal the underlying submucosal tissue layer 30 from the surrounding environment, e.g., stomach, esophageal or bowel contents, bacteria or other harmful or infectious elements (FIG. 5).

Sealant material 40′ may be formulated from a material that expands during the transition from an uncured configuration to a cured configuration further lifting and separating the tissue layers 20, 30 and facilitating resection of excision of the abnormal tissue growth 10. It is envisioned that a sealant 40′ may also be formulated as a substantially solid material or fluid-like material to accomplish the same or similar purpose. It is also envisioned that the sealant 40′ may be formulated from a material that does not necessarily cure but acts to readily separate and seal the two tissue layers 20 and 30 immediately upon injection.

The sealant material 40′ may be curable upon selective application of temperature, electrosurgical energy, ultrasonic energy, light and/or combinations thereof. For example, the sealant material 40′ may be formulated with any of the following characteristics or combinations of characteristics:

1) to cure on contact or exposure to the submucosal layer 30;

2) to cure by a change in temperature, i.e., upon contact with the higher body temperature of the submucosal layer 30 or additional temperature or heat applied relative to the tissue site;

3) to cure upon application of energy, e.g., RF, ultrasonic or microwave; and/or

4) to cure upon application of light, e.g., laser, ultraviolet, etc.

During the curing process, and especially when heat or electrical application is applied to resect or excise the abnormal tissue growth 10, the sealant material 40′ may be formulated to provide thermal insulation for adjacent tissue layers, e.g., mucosal layer 20 or submucosal layer 30. More particularly, the sealant material 40′ may be formulated to regulate, e.g., maximize, minimize and/or maintain the sealant's material 40′ relative impedance during the application of thermal or electrosurgical energy to protect surrounding tissue layers.

The sealant material 40′ may include collagen, liposomes, elastin and/or combinations thereof. Moreover, the sealant may be formulated to include a collagen or elastin (or combination) material that is designed to react or cooperate with native collagen to form a high strength seal once cured. The sealant material 40′ may also be formulated to include an antibiotic solution, an antibacterial solution, hemostatic solution, antibodies and/or combinations thereof. The term “hemostatic solution” is defined herein to mean any formulation or solution which causes or facilitates hemostasis after an excision.

The present disclosure also relates to a method for excising abnormal tissue and includes the steps of: providing a sealant material 40′ adapted to transition from an uncured configuration, wherein the sealant material 40′ is allowed to be injected between an abnormal tissue growth 10 and underlying tissue, e.g., submucosal tissue layer 30, and a cured configuration, wherein the sealant material 40″ acts as a substantially solid barrier between the abnormal tissue growth 10 and underlying tissue 30. The method may also includes the steps of: injecting the sealant material 40′ between the abnormal tissue growth 10 and the underlying tissue 30 to initially separate the abnormal tissue growth 10 relative to the underlying tissue 30 and allowing the sealant material 40′ to cure (now sealant material 40″). Thereafter, the abnormal tissue growth 10 is excised via a snare 60, wire loop, forceps, knife, scissors and/or combinations thereof. After excision of the target tissue 10, the sealant material 40″ acts to seal the underlying tissue 30 from the surrounding environment.

As mentioned above, the sealant material 40′ may be formulated to expand during the transition from an uncured configuration to a cured configuration. Moreover, the step of curing the sealant material 40′ may include the step of selectively applying temperature, electrosurgical energy, ultrasonic energy and/or light and/or combinations thereof to cure the sealant material 40′.

Also as mentioned above, the sealant material 40′ may be formulated to include collagen, liposomes, elastin and/or combinations thereof. The sealant material 40′ may also be formulated to include an antibiotic solution, an antibacterial solution, antibodies and/or combinations thereof. Moreover, the sealant material 40′ may be formulated to include a material that regulates the sealant's material 40′ impedance during excision (e.g., the application of energy) to regulate the temperature of surrounding tissue layers.

From the foregoing and with reference to the various figure drawings, those skilled in the art will appreciate that certain modifications can also be made to the present disclosure without departing from the scope of the same. While only one particularly-envisioned embodiment of the disclosure has been shown in the drawings, it is not intended that the disclosure be limited thereto, as it is intended that the disclosure be as broad in scope as the art will allow and that the specification be read likewise. Therefore, the above description should not be construed as limiting, but merely as exemplifications of particular embodiments. Those skilled in the art will envision other modifications within the scope and spirit of the claims appended hereto.

What is claimed is:

1. A system for excision of abnormal tissue, the system comprising:
a sealant material having an uncured configuration allowing the sealant to be readily injected between target tissue and underlying tissue and a cured configuration wherein the sealant material acts as a substantially solid barrier between the target tissue and underlying tissue, the sealant material acting initially to separate the target tissue relative to the underlying tissue to facilitate excision of the target tissue via at least one of a snare, wire loop, forceps, knife, scissors and combinations thereof, wherein after excision of the target tissue, the sealant material acting to seal the underlying tissue from the surrounding environment.

2. A system according to claim 1 wherein during transition from the uncured configuration to the cured configuration, the sealant material expands to separate the target tissue from the underlying tissue.

3. A system according to claim 1 wherein the sealant material is curable upon selective application of at least one of temperature, electrosurgical energy, ultrasonic energy and light and combinations thereof.

4. A system according to claim 3 wherein the sealant material provides thermal insulation for adjacent tissue layers during electrical activation for at least one of curing and excision purposes.

5. A system according to claim 1 wherein the sealant material includes at least one of collagen, liposomes, elastin and combinations thereof.

6. A system according to claim 1 wherein the sealant material includes collagen, which is designed to react with native collagen to form a high strength seal.

7. A system according to claim 1 wherein the sealant material is formulated to regulate the sealant material's impedance during the application of energy to regulate the temperature of surrounding tissue layers.

8. A system according to claim 1 wherein the sealant material includes at least one of an antibiotic solution, an antibacterial solution, hemostatic solution, antibodies and combinations thereof.

9. A system for use with the excision of abnormal tissue, the patch comprising:
a sealant material having an uncured configuration allowing the sealant material to be readily injected through an endoscopic instrument adjacent abnormal tissue and between mucosa tissue and submucosa tissue and a cured configuration wherein the sealant material acts as a substantially solid barrier between the mucosa tissue and submucosa tissue, the sealant material acting initially to separate the abnormal tissue and mucosa tissue relative to the submucosa tissue to facilitate excision of the abnormal tissue via at least one of a snare, wire loop, forceps, knife, scissors and combinations thereof, wherein after excision of the abnormal tissue, the sealant material acting to seal the submucosa tissue from the surrounding environment.

10. A method for excising abnormal tissue, the method comprising the steps of:
providing a sealant material having an uncured configuration;
injecting the sealant material in the uncured configuration between target tissue and underlying tissue to initially separate the target tissue relative to the underlying tissue;
allowing the sealant material to cure to a cured configuration; and
excising the target tissue, wherein after excision of the target tissue, the sealant material acts to seal the underlying tissue.

11. A method according to claim 10 wherein the sealant material expands during the transition from the uncured configuration to the cured configuration.

12. A method according to claim 10 wherein the step of allowing the sealant material to cure includes selectively applying at least one of temperature, electrosurgical energy, ultrasonic energy and light to cure the sealant material.

13. A method according to claim 10 wherein the sealant material includes at least one of collagen, liposomes and elastin.

14. A method according to claim 10 wherein the sealant material includes a material that regulates the sealant material's impedance during the application of energy to regulate the temperature of surrounding tissue layers.

15. A method according to claim 10 wherein the sealant material includes at least one of an antibiotic solution, an antibacterial solution, hemostatic solution and antibodies.

16. A method for excising abnormal tissue, the method comprising the steps of:
providing a substantially liquid sealant material operably transitionable between an uncured configuration and a cured configuration when exposed to at least one of temperature, electrosurgical energy, ultrasonic energy and light, the substantially liquid sealant material being formulated with at least one of an antibiotic solution, an antibacterial solution, hemostatic solution and antibodies;
injecting the substantially liquid sealant material in an uncured configuration between target tissue and underlying tissue to initially separate the target tissue relative to the underlying tissue;
allowing the substantially liquid sealant material to transition from the uncured configuration to the cured configuration by selectively exposing the substantially liquid sealant material to at least one of temperature, electrosurgical energy, ultrasonic energy and light; and
excising the target tissue, wherein after excision of the target tissue, the sealant material acts to seal the underlying tissue.
